(19) United States
(12) Patent Application Publication
(10) Pub. No.: US 2003/0060714 A1
(43) Pub. Date: Mar. 27, 2003

(54) Medical ultrasound transducer with interchangeable handle

(76) Inventors: Richard W. Henderson, Fremont, CA (US); Joseph J. Molinari, Redwood City, CA (US)

Correspondence Address: BRINKS HOFER GILSON & LIONE, P.O. BOX 10395, CHICAGO, IL, 60611, US

(21) Appl. No.: 09/963,097

(22) Filed: Sep. 24, 2001

(51) Int. Cl. A61B 8/14

(52) U.S. Cl. 600/459

(57) ABSTRACT

A medical ultrasonic transducer includes a transducer body that releasably carries a handle. The handle forms a gripping surface for the user, and multiple handles are provided, all fitting on the same transducer body but sized differently to accommodate differently-sized hands. Individual handles can be removed and replaced in a clinical setting to modify the size and/or shape of the gripping surface as appropriate for an individual user.

### BACKGROUND

The present invention relates to ultrasound transducers for medical ultrasonic imaging systems, and in particular to improved handles for such transducers.

In the past, ultrasound transducers used with medical imaging systems have been provided with external covers that serve as the handle by which the transducer is gripped by the user. Often, large transducers are provided with large covers that provide large handle grips, and small transducers are provided with small covers that provide small handle grips. The result of this prior-art approach is that users are often required to grip and use transducers that do not fit their hands properly. This can lead to discomfort and inefficiency.

U.S. Pat. No. 5,897,503 discloses an ultrasound transducer having an elastomeric cover that may be customized for the individual user. However, once customized for one user, the transducer handle may not be optimized in size or shape for other users.

### SUMMARY

By way of general introduction, the preferred embodiments described below include a transducer body that carries a transducer array, and a plurality of transducer handles. Each of the handles has a respective gripping portion, and at least two of the gripping portions differ in size. Individual handles can be removed from the transducer body and replaced by other transducer handles in a clinical setting. In this way, the ultrasound transducer can readily be modified in the clinical setting to provide a handle that is ergonomically well-suited to the needs of a particular user.

This section has been provided by way of general introduction, and it should not be used to narrow the scope of the following claims.

### BRIEF DESCRIPTION OF THE DRAWINGS

FIG. 1 is a perspective view of an ultrasonic transducer that includes a partially disassembled tubular handle.

FIG. 2 is a perspective view of three interchangeable handles that can be mounted on the transducer of FIG. 1 in a clinical setting.

FIG. 3 is a perspective view of another transducer that includes a pair of handle shells interconnected by a hinge.

FIG. 4 is a perspective view of a third transducer that includes a pair of handle shells held together by threaded fasteners.

FIG. 5 is a flow chart of a method for using the transducers of FIGS. 1-4.

### DETAILED DESCRIPTION OF THE PRESENTLY PREFERRED EMBODIMENTS

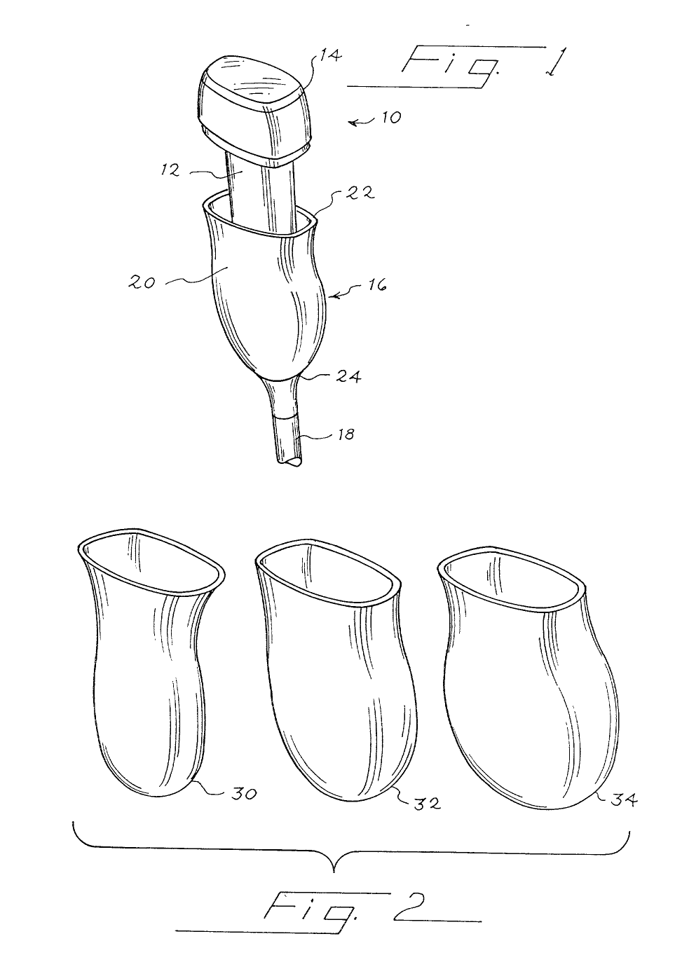

Turning now to the drawings, FIG. 1 shows a perspective view of an ultrasound transducer 10. The ultrasound transducer 10 includes a transducer body or core 12, and the body 12 carries an array 14 of transducer elements. The body 12 and the array 14 form a sealed, self-contained unit that performs all of the ultrasound functions of the transducer 10, but that is not ergonomically optimized to fit the hand of the user. The body 12 is connected to a cable 18 that includes a distal end (not shown). This distal end typically includes an electrical connector (not shown) for connecting the cable 18 to an ultrasonic imaging system (not shown).

The body 12, the array 14, and the cable 18 can vary widely, and many prior-art techniques can be used for manufacturing these elements. For example, the array 14 may include a 1, 1.5, or 2-dimensional array, and any suitable transducer material can be used, including a variety of piezoelectric materials. The array 14 may be releasably connected to the body 12, as described in U.S. Pat. Nos. 5,820,549 and 5,617,866.

As shown in FIG. 1, the transducer 10 also includes a handle 16 that in this embodiment is tubular in shape. The handle 16 defines a generally rigid gripping portion 20 that is bounded by an upper rim 22 and a lower rim 24. The rims 22, 24 are configured to fit against the mating structures of the body 12, and the rims 22, 24 can be taken as an example of mechanical fasteners. In FIG. 1, the handle 16 is shown in a partially disassembled state, in which the handle 16 has been moved away from the array 14 along the cable 18. In actual use, the handle 16 would be moved toward the array 14 until the rims 22, 24 engage corresponding features of the body 12. In this position, the handle 16 merges smoothly and continuously with the forward portion of the transducer 10, and the body 12 is covered and hidden by the handle 16.

The handle 16 is designed to be readily removable from the body 12. For example, the array 14 may be disconnected from the body 12 to allow removal of the handle 16 (See U.S. Pat. Nos. 5,820,549 and 5,617,866).

Alternatively, a connector (not shown) can be provided between the body 12 and the cable 18, and the cable 18 may be disconnected from the body 12 to allow removal of the handle 16.

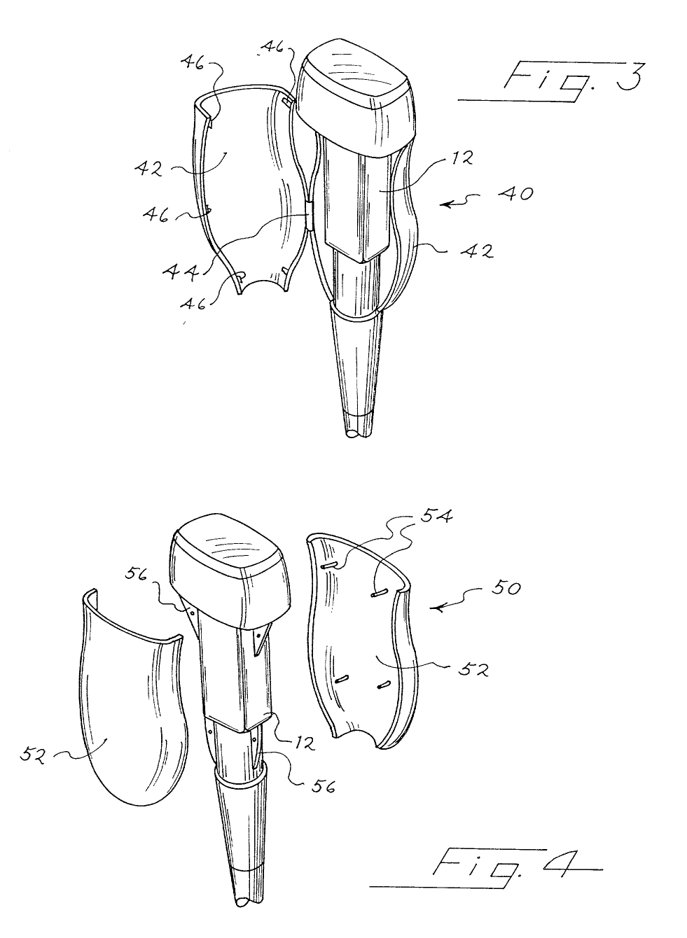

As shown in FIG. 2, the transducer 10 can be included in a transducer set that includes multiple handles. In this case, three handles 30, 32, 34 are shown. Any one of the three handles 30, 32, 34 can be selected, moved over the body 12, and then fastened in place around the body 12. As shown in FIG. 2, the three handles 30, 32, 34 differ in size of the respective gripping portions. The handle 30 is suited for a user with a small hand; the handle 32 is suited for a user with a medium hand; and the handle 34 is suited for a user with a large hand. All of the handles 30, 32, 34 define upper rims that are identical to one another and lower rims that are identical to one another. This allows all of the handles 30, 32, 34 to fit interchangeably on the transducer 10.

The handles 16, 30, 32, 34 can be secured in place on the body 12 using any desired type of fastener, including snap-lock fasteners, threaded fasteners and the like. Regardless of the fasteners used, it is preferred that the fastener be of the type that can be readily released and attached in a clinical setting. This allows the user to customize the size of the handle of the transducer 10 by selecting the appropriate handle and installing the selected handle on the body 12.

As used herein, the term “clinical setting” is intended broadly to encompass offices, clinics, hospitals and maintenance areas associated with offices, clinics, and hospitals. The term “clinical setting” does not include a manufacturing setting such as the plant where the transducer 10 was manufactured.

FIG. 3 shows another handle 40 that includes two shells 42 mounted together at a hinge 44. The hinge 44 may, for example, be a living hinge. The handle 40 includes fasteners 46 that in this example take the form of snap-lock fasteners that releasably hold the shells 42 in position around the body 12. A user can readily replace the handle 40 with another similar handle having a differently sized gripping portion by opening the fasteners 46, moving the shells 44 away from each other, removing the original handle 40, and then reversing these procedures with another handle that is similar to the handle 40 but that differs in the size or shape of the gripping portion.

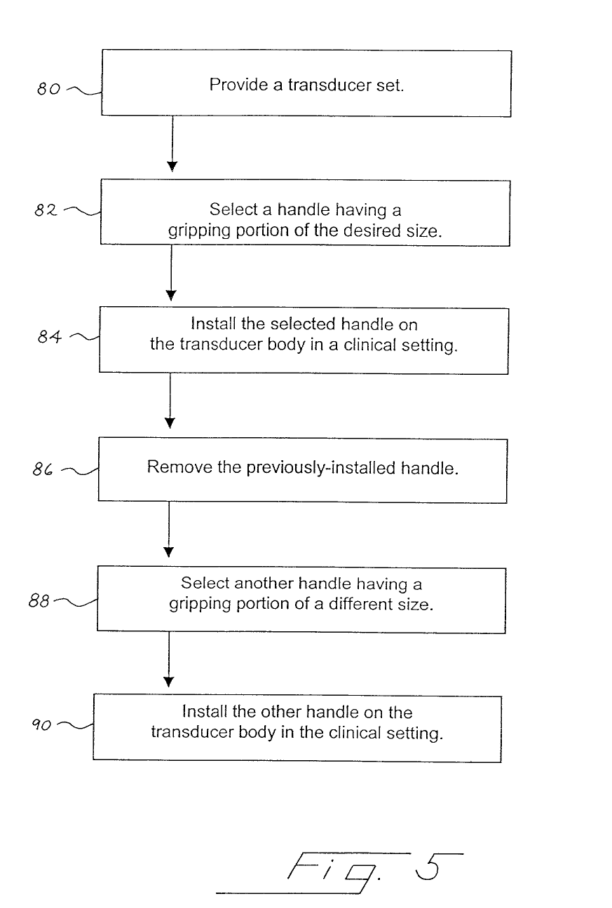

FIG. 4 shows another handle 50 that in this case includes two shells 52. The shells 52 can be releasably secured together by fasteners 54 that in this example take the form of threaded fasteners or fasteners that snap between an over-center, locked position and a release position. As shown in FIG. 4, the body 12 in this example includes mounting flanges 56 that receive the fasteners 54 and assist in retaining the shells 52 in position when the fasteners 54 are tightened. As with other illustrated embodiments, the handle 50 can readily be removed and replaced in a clinical setting with a similar handle having a gripping portion of a differing size.

The handles described above can take many forms. For example, the handle may be disposed at one side or one end of the body, such that the handle does not extend around the body. The handles can be formed of any suitable material, such as various polycarbonates and polysulfones. For example, the handles 16, 30, 32, 34 and the shells 42, 52 may be molded from a rigid plastic such as the polyphenylsulfone sold by Amoco under the trade name Radel. Though the handles are generally pre-formed in the desired shape and generally rigid, they may include elastomeric inserts, surfaces, or other portions, as described in U.S. Pat. No. 5,897,503.

FIG. 5 illustrates a method that can be performed using any of the transducers and handles described above. In block 80, a transducer set is provided. As used herein, the term “transducer set” includes a transducer body 12, an array 14, and multiple handles. The handles of the set include at least two handles that differ in the sizes of the respective gripping portions.

In block 82, a handle is selected having a gripping portion of the desired size. The selected handle may, for example, be the handle 30 of FIG. 2.

In block 84, the selected handle is installed on the transducer body in a clinical setting. As pointed out above, the handles and the fasteners have been designed such that the transducer does not have to be returned to the manufacturer to install the handle.

In block 86, the previously-installed handle is removed from the transducer body, and in block 88 another handle is selected having a different gripping portion. For example, the other handle selected in block 88 can be the handle 34 of FIG. 2.

In block 90, the other handle selected in block 88 is installed on the transducer body in a clinical setting. As explained above, this installation can readily be accomplished in a clinical setting without exotic tools or assembly processes.

From the foregoing, it should be apparent that a medical ultrasound transducer has been described having a modular handle. This modular handle allows any one of several handles that differ in size to be selected and installed in a clinical setting. In this way, the transducer can be customized in a clinical setting to provide different handles for different users. Various users can therefore operate the same transducer in a comfortable and efficient manner.

It should be understood that a wide range of changes and modifications can be made to the preferred embodiments described above. The handles and related fasteners can be designed differently than in the illustrated embodiments. For example, a handle that slides in place over the transducer body may be held in place by fasteners such as dovetail-shaped guides. Also, the handle fasteners can include hook-and-loop fasteners, snap fasteners, adhesive fasteners and the like. Snap fasteners may rely on the elastic properties of the materials from which they are formed to create a snap latch. Other mechanical fasteners can be used such as mechanical latches that latch and unlatch under operator control. For example, a button release may be provided to allow a user to retract a spring-loaded bolt or pin that mechanically secures the handle in place on the body. If desired, the handle fasteners may be installed on the body such that the same handle fasteners secure different handles in place at different times.

The modular handles described above can be made to be disposable to eliminate cleaning issues, or they can be readily cleaned by removing them from the transducer and cleaning them in the removed state.

The foregoing detailed description has discussed only a few of the many forms that this invention can take. For this reason, this detailed description is intended by way of illustration and not limitation. It is only the following claims, including all equivalents, that are intended to define the scope of this invention.

1. An ultrasound transducer set comprising:
a transducer body;
a transducer array carried by the body;
a plurality of transducer handles, each of the transducer handles comprising a respective pre-formed gripping portion; and
at least one handle fastener configured to releasably secure the respective handle to the body such that the handles are exchangeable on the body in a clinical setting;
the transducer handles comprising at least two gripping portions that differ in size.

2. The invention of claim 1 further comprising:
a transducer cable coupled with the transducer array and carried by the body independently of all of the handles.

3. The invention of claim 1 wherein each handle is generally tubular in shape.

4. The invention of claim 1 wherein each handle comprises a pair of shells, and wherein each handle fastener secures the shells together around the body.

5. The invention of claim 4 wherein each handle further comprises at least one hinge that connects the respective pair of shells together on one side.

6. The invention of claim 1 wherein each handle fastener comprises a respective threaded fastener.

7. The invention of claim 1 wherein each handle fastener comprises a respective snap-lock fastener.

8. The invention of claim 1 wherein each handle comprises a substantially rigid portion configured to surround the body.

9. A method for customizing an ultrasonic transducer for a hand of a user, said method comprising:
(a) providing an ultrasound transducer set comprising:
a transducer body;
a transducer array carried by the body;
a plurality of transducer handles, each of the transducer handles comprising a respective pre-formed gripping portion; and
at least one handle fastener configured to releasably secure the respective handle to the body such that the handles are exchangeable on the body in a clinical setting;
the transducer handles comprising at least two gripping portions that differ in size;
(b) selecting one of the handles that fits the hand of the user; and
(c) installing the handle selected in (b) on the transducer body in a clinical setting.

10. The method of claim 9 further comprising:
(d) removing the handle installed in (c) from the transducer body in the clinical setting;
(e) selecting another one of the handles, different than the handle selected in (b); and
(f) installing the handle selected in (e) on the transducer body in the clinical setting.
